(19) United States
(12) Patent Application Publication
(10) Pub. No.: US 2024/0315612 A1
(43) Pub. Date: Sep. 26, 2024

(54) MEASURING DEVICE

(71) Applicant: TAIYO YUDEN CO., LTD., Tokyo (JP)

(72) Inventors: Katsuhiro OYAMA, Gunma (JP); Takayuki SEKIGUCHI, Gunma (JP)

(73) Assignee: TAIYO YUDEN CO., LTD., Tokyo (JP)

(21) Appl. No.: 18/680,223

(22) Filed: May 31, 2024

Related U.S. Application Data
(63) Continuation of application No. PCT/JP2022/039573, filed on Oct. 24, 2022.

(30) Foreign Application Priority Data
Dec. 3, 2021 (JP) 2021-197333

(51) Int. Cl. A61B 5/145; A61B 5/00; A61B 5/1455

(52) U.S. Cl. CPC A61B 5/14532; A61B 5/1455; A61B 5/681

(57) ABSTRACT

A measuring device includes a first light emitting section that emits first light; a light collecting section that has a first surface, a second surface that faces the first surface and that has a larger area than the first surface, and a wall surface that connects the first surface and the second surface and that allows the first light to pass therethrough; a light receiving section that is disposed on the first surface to receive the first light irradiated onto a living body from the second surface and returned from the living body; and a calculation section that receives an output from the light receiving section to calculate numerical information regarding the living body.

### CROSS-REFERENCE TO RELATED APPLICATIONS

This application is a continuation of PCT International Application No. PCT/JP2022/039573 filed on Oct. 24, 2022, which designates the United States, incorporated herein by reference, and which claims the benefit of priority from Japanese Patent Application No. 2021-197333, filed on Dec. 3, 2021, incorporated herein by reference.

### BACKGROUND OF THE INVENTION

### Technical Field

The present invention relates to a measuring device.

### Background Art

Some biological numerical information, such as glucose concentration in blood, is measured using blood sampling. However, in recent years, optical measurement techniques that do not require blood sampling have been developed in order to avoid concerns about the physical burden on patients and infectious diseases caused by blood sampling (see, for example, Japanese Patent Application Laid-Open Publication No. 2011-62335).

### SUMMARY OF THE INVENTION

For example, the concentration of glucose contained in the interstitial fluid of the dermis is correlated with the concentration of glucose in the blood, and if the concentration of glucose contained in the interstitial fluid of the dermis can be detected, the concentration of glucose in the blood can be estimated. According to optical measurement technology, the surface of a living body is irradiated with light of a specific wavelength that is easily absorbed by glucose. Then, the concentration of glucose contained in the interstitial fluid of the dermis is detected based on the intensity of the light that returns from the skin via the dermis.

When determining the concentration of glucose in blood by the above method, it is desirable that the amount of return light from the living body that enters the light receiving section relative to the amount of light emitted by the light emitting section, that is, the light utilization efficiency, be as large as possible. This is because when the light utilization efficiency is low, in order to increase the amount of detected return light to a predetermined amount or more, costly measures such as increasing the number of one or both of the light emitting section and the light receiving section are required.

An object of the present invention is to provide a measuring device with improved light utilization efficiency.

Additional or separate features and advantages of the invention will be set forth in the descriptions that follow and in part will be apparent from the description, or may be learned by practice of the invention. The objectives and other advantages of the invention will be realized and attained by the structure particularly pointed out in the written description and claims thereof as well as the appended drawings.

To achieve these and other advantages and in accordance with the purpose of the present invention, as embodied and broadly described, in one aspect, the present disclosure provides a measuring device that includes a first light emitting section that emits first light; a light collecting section that has a first surface, a second surface that faces the first surface and that has a larger area than the first surface, and a wall surface that connects the first surface and the second surface and that allows the first light to pass therethrough; a light receiving section that is disposed on the first surface to receive the first light irradiated onto a living body from the second surface and returned from the living body; and a calculation section that receives an output from the light receiving section to calculate numerical information regarding the living body.

It is to be understood that both the foregoing general description and the following detailed description are exemplary and explanatory, and are intended to provide further explanation of the invention as claimed.

### BRIEF DESCRIPTION OF THE DRAWINGS

FIG. 1 is a diagram for explaining the absorption characteristics of infrared light by glucose and albumin, which is a main component of the skin.

FIG. 2 is a schematic diagram for explaining variations in the path of light irradiated onto a living body.

FIG. 3 is a diagram for explaining the distribution of the intensity of light reflected within the dermis and returned to the skin surface.

FIG. 4 is a schematic diagram showing an example of the configuration of the glucose measuring device according to an embodiment.

FIG. 5 is an external view of the trapezoidal prism according to the embodiment viewed from the top side.

FIG. 6 is a cross-sectional view of the trapezoidal prism according to the embodiment taken along the cutting line VI-VI shown in FIG. 5.

FIG. 7 is a cross-sectional view of the trapezoidal prism according to the embodiment taken along the cutting line VII-VII shown in FIG. 5.

FIG. 8 is a schematic diagram showing an example of the configuration of the light receiving module according to the embodiment.

FIG. 9 is a schematic diagram for explaining an example of the arrangement relationship between the trapezoidal prism according to the embodiment and components around it according to the embodiment.

FIG. 10 is a diagram showing an example of the relationship between the height H of the trapezoidal prism and the light utilization efficiency according to the embodiment.

FIG. 11 is a perspective view illustrating an example of a structure that supports the trapezoidal prism and surrounding components according to the embodiment.

FIG. 12 is a cross-sectional view of the structure shown in FIG. 11 taken along cutting line XII-XII.

FIG. 13 is a flowchart illustrating an example of the operation of the glucose measuring device according to an embodiment.

FIG. 14 is a top view of a hood as another example of the light collecting section according to an embodiment.

FIG. 15 is a bottom view of the hood as another example of the light collecting section according to the embodiment.

FIG. 16 is a side view of the hood as another example of the light collecting section according to the embodiment.

FIG. 17 is a cross-sectional view of the hood as another example of the light collecting section according to the embodiment, taken along the cutting line XVII-XVII shown in FIG. 15.

FIG. 18 is a schematic diagram for explaining an example of the arrangement relationship between the hood as another example of the light collecting section according to the embodiment and components around the hood.

### DETAILED DESCRIPTION OF EMBODIMENTS

The measuring device according to the present invention is an optical measuring device that measures numerical information regarding a living body. The numerical information regarding the living body is, for example, the concentration of glucose in the blood and/or the pulse wave (that may relate to heartbeat). The living body may be a human or a non-human living body such as a livestock animal. As an example, a glucose measuring device that measures the concentration of glucose in human blood will be described. Note that the optical measuring device according to the present invention is not limited to a glucose measuring device. For example, the optical measuring device according to the present invention can also be applied to a pulse wave measuring device that measures the pulse wave of a living body. The optical measuring device according to the present invention can be implemented in a wristwatch, or can be configured as a wristwatch-type device. The optical measuring device according to the present invention can also be applied to a measuring device that measures the concentration of other specific components contained in blood.

A glucose measuring device according to an embodiment will be described below with reference to the drawings. Note that the present invention is not limited to this embodiment.

### (Wavelength of Light Used in Glucose Measuring Device 1 According to the Embodiment)

The glucose measuring device 1 according to the embodiment irradiates the epidermis of the human body with infrared light and measures the concentration of glucose in the blood based on the intensity of the light returning from the epidermis of the living body.

As mentioned above, there is a correlation between the concentration of glucose contained in the interstitial fluid of the dermis and the concentration of glucose in the blood. Therefore, in the embodiment, the concentration of glucose contained in the interstitial fluid of the dermis is the primary target of measurement.

In order to accurately detect the concentration of glucose contained in the interstitial fluid of the dermis, the light used for detection must not only be easily absorbed by glucose, but also have properties that make it difficult to be absorbed by the constituent components of the skin so as to penetrate the epidermis and propagate to the dermis.

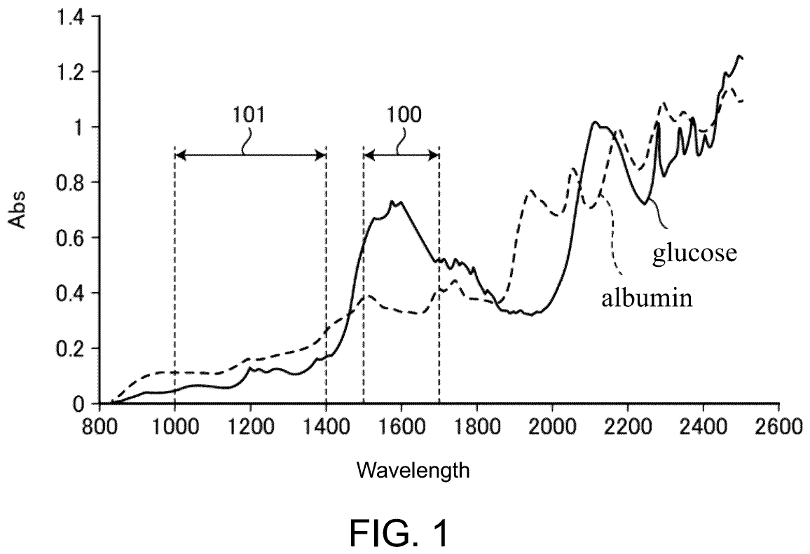

FIG. 1 is a diagram for explaining the absorption characteristics of infrared light by glucose and albumin, the latter of which is a main component of the skin. The horizontal axis of this figure shows the wavelength of infrared light, and the vertical axis shows absorbance (Abs). From this figure, it can be seen that light in the band of 1500 nm to 1700 nm (the range 100 in the figure) is hardly absorbed by albumin and easily absorbed by glucose.

Therefore, light with a wavelength selected from the range of 1500 nm to 1700 nm is used to detect glucose. The light used for detecting glucose, having a wavelength selected from the range of 1500 nm to 1700 nm, is referred to as the “first light.”

Note that the dermis exists directly below the epidermis. The thickness of the epidermis may vary depending on the body part. Furthermore, even in the same region, there are differences among individuals in the thickness of the epidermis. In order to reduce the influence of differences in epidermal thickness on detected values, in addition to the first light, light having a wavelength selected from a range different from the range of 1500 nm to 1700 nm is used. This light will be referred to as the “second light.” The second light has a wavelength selected from the range of, for example, 1000 nm to 1400 nm (the range 101 in the figure), which is absorbed to some extent by albumin and has flat absorption characteristics for both albumin and glucose.

### (Regarding the Reflection of Incident Light on the Dermis)

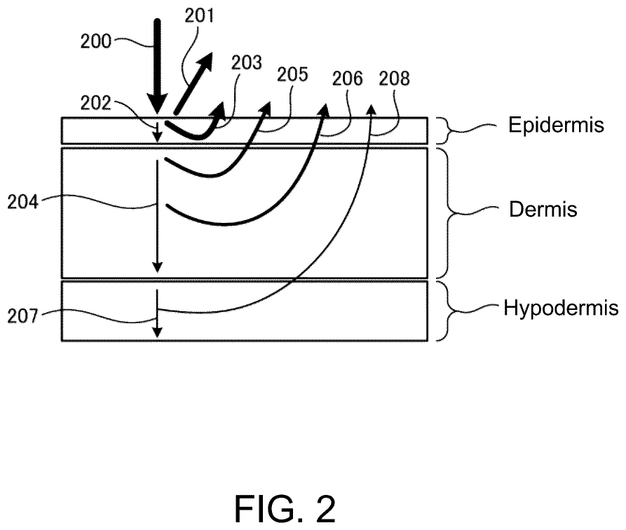

FIG. 2 is a schematic diagram for explaining variations in the path of light irradiated onto a living body. The light irradiated here is, for example, light in a band of 1500 nm to 1700 nm. The skin of the human body is covered on the outermost layer by the epidermis. The dermis lies below the epidermis. Further below the dermis layer is hypodermis. In the case of a human forearm, the thicknesses of the epidermis layer, dermis layer, and hypodermis are approximately 0.2 mm, 2 mm, and 0.9 m, respectively.

When a living body is irradiated with light 200, as shown by light 201, a portion of the light 200 is reflected on the surface of the epidermis. The remaining light 200 irradiated to the living body penetrates the epidermis, and a part of the light 202 that penetrates the epidermis is reflected within the epidermis and goes back outside as shown by light 203.

The remaining part of the light 202 that has penetrated the epidermis layer penetrates the dermis below the epidermis layer. A portion of the light 204 that has entered the dermis is reflected within the dermis and returns outside the living body, as shown by light 205 and light 206.

The remaining part of the light 204 that has penetrated the dermis penetrates the hypodermis below the dermis. A portion of the light 207 that has entered the hypodermis is reflected within the hypodermis and returned outside the living body, as shown by light 208.

If we can collect the reflected light 205 and 206 as much as possible, which is the light that is reflected back within the dermis layer among these lights, light 201, light 203, light 205, light 206, and light 208, the concentration of glucose in the dermis can be detected with high accuracy.

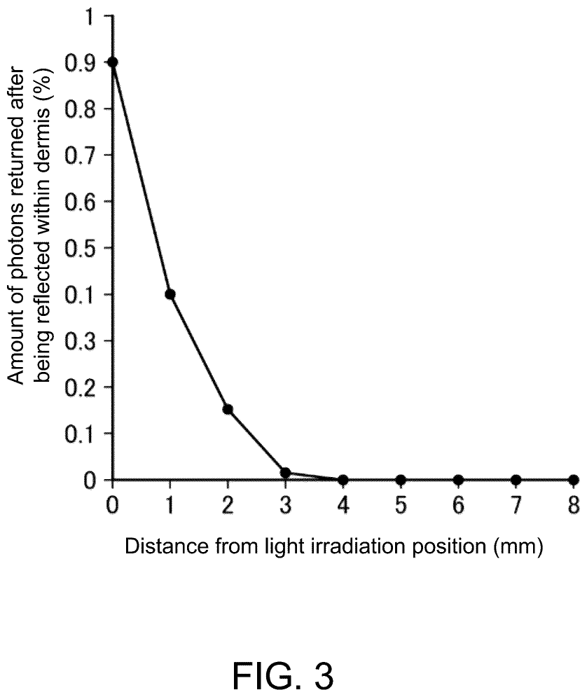

FIG. 3 is a diagram for explaining the distribution of the intensity of light reflected within the dermis and returned with respect to the distance from the skin surface. The horizontal axis of this figure indicates the distance from the origin O on the skin surface, where the origin O is the position on the skin surface where the light is irradiated. The vertical axis indicates the ratio of the amount of photons reflected within the dermis layer, such as light 205 and light 206, when the amount of light irradiated to the origin O is 100%. Note that this figure was obtained by a simulation of light propagation using the Monte Carlo method. As the optical values, the optical values of the composition of a general human body were used.

As shown in FIG. 3, the intensity of the light reflected within the dermis and returned is strongest at the origin O, and decreases as it moves away from the origin O. At a position 3 mm away from the origin O, the intensity of the light reflected within the dermis is very small, and at a position 4 mm away from the origin O, the intensity of the light reflected within the dermis is almost zero.

In other words, the intensity of the light that is reflected within the dermis and returned is the strongest at the origin O, and the light that is reflected within the dermis and returned is distributed over a distance up to 4 mm from the origin O.

In the embodiment, based on the above findings, the glucose measuring device 1 includes a trapezoidal prism (trapezoidal prism 2) as a light collecting section that is pressed against the skin surface and collects light returned from the human body so that the light reflected within the dermis and returned can be efficiently collected. Details of the trapezoidal prism 2 will be described later.

### (Configuration of the Glucose Measuring Device According to an Embodiment)

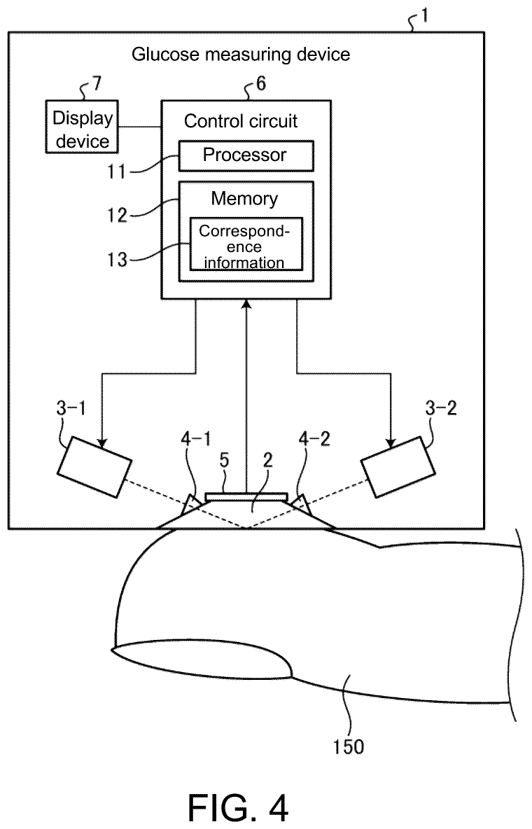

FIG. 4 is a schematic diagram showing an example of the configuration of the glucose measuring device 1 according to an embodiment. The glucose measuring device 1 includes a trapezoidal prism 2, a first light emitting element 3-1, a second light emitting element 3-2, a first triangular prism 4-1, a second triangular prism 4-2, a light receiving module 5, a control circuit 6, and a display device 7.

The first light emitting element 3-1 emits first light. As the first light emitting element 3-1, for example, a laser diode that emits light with a wavelength of 1550 nm, which is widely used in the field of infrared optical communication, can be employed as the first light emitting element 3-1.

Note that the first light emitting element 3-1 is an example of a first light emitting section. Any light source can be employed as the first light emitting section as long as it emits a wavelength selected from the range 100 of 1500 nm to 1700 nm.

The first light emitting element 3-1 includes a collimating lens inside so that it can emit collimated light. Note that the collimating lens may be provided outside the first light emitting element 3-1.

The second light emitting element 3-2 emits second light. As the second light emitting element 3-2, for example, a laser diode that emits light with a wavelength of 1310 nm, which is widely used in the field of infrared communication, can be employed as the second light emitting element 3-2.

Note that the second light emitting element 3-2 is an example of a second light emitting section. Any light source can be employed as the second light emitting section as long as it emits light of a wavelength selected from a range different from the range 100 of 1500 nm to 1700 nm.

The second light emitting element 3-2 is equipped with a collimating lens inside so that it can emit collimated light. Note that the collimating lens may be provided outside the second light emitting element 3-2.

The trapezoidal prism 2 is an optical element that is made of a medium that transmits predetermined light and that has two parallel surfaces and four side walls that connect the two parallel surfaces and that are inclined with respect to the two parallel surfaces. Here, the predetermined lights are the lights used for measurement, that is, the first light and the second light. The medium that transmits the predetermined light is, for example, a light-transmissive material such as glass or transparent plastic. The trapezoidal prism 2 is an example of a light collecting section and an example of the “first optical element.”

For convenience, the surface with the larger area of the two parallel surfaces of the trapezoidal prism 2 is referred to as the bottom surface. The surface with a smaller area of the two parallel surfaces of the trapezoidal prism 2 is referred to as the top surface. Note that as long as the side walls (side walls 2c and 2d, which will be described later) on which the light used for measurement is incident among the four side walls of the trapezoidal prism 2 are inclined with respect to the two parallel surfaces, the other side walls (side wall 2e and side wall 2f described below) do not necessarily have to be inclined with respect to two parallel planes. The other side walls may be perpendicular to the two parallel surfaces.

When measuring the glucose concentration, the human body 150 is pressed against the bottom surface of the trapezoidal prism 2. Note that in the example of this figure, the human body 150 is a finger. The part of the human body 150 that is pressed against the bottom surface of the trapezoidal prism 2 is not limited to the fingers.

The first triangular prism 4-1 and the second triangular prism 4-2 are optical elements having the shape of a triangular prism and made of a medium that transmits light used for measurement. The refractive index of the first triangular prism 4-1 and the second triangular prism 4-2 is equal to the refractive index of the trapezoidal prism 2. The medium that transmits the predetermined light is, for example, a light-transmissive material such as glass or transparent plastic. Note that the first triangular prism 4-1 is an example of the “second optical element.”

The first light emitted by the first light emitting element 3-1 enters the trapezoidal prism 2 from the side wall surface of the trapezoidal prism 2 via the first triangular prism 4-1, and reaches the bottom surface of the trapezoidal prism 2. When the human body 150 is pressed against the bottom surface of the trapezoidal prism 2, the first light is irradiated onto the human body 150 from the bottom surface of the trapezoidal prism 2, and the first light returned from the human body 150 is incident upon the bottom surface of the trapezoidal prism 2. The first light returning from the human body 150 that entered the trapezoidal prism 2 is collected at the top surface of the trapezoidal prism 2 with multiple reflections within the trapezoidal prism 2 or directly without the multiple reflections.

The second light emitted by the second light emitting element 3-2 enters the trapezoidal prism 2 from the side wall surface of the trapezoidal prism 2 via the second triangular prism 4-2, and reach the bottom surface of the trapezoidal prism 2. When the human body 150 is pressed against the bottom surface of the trapezoidal prism 2, the second light is irradiated onto the human body 150 from the bottom surface of the trapezoidal prism 2, and the second light returned from the human body 150 is incident upon the bottom surface of the trapezoidal prism 2. The second light returning from the human body 150 that has entered the trapezoidal prism 2 is collected at the top surface of the trapezoidal prism 2 with multiple reflections within the trapezoidal prism 2 or directly without the multiple reflections.

A light receiving module 5 is arranged on the top surface of the trapezoidal prism 2. The light receiving module 5 is an example of a light receiving section. As the light receiving section, any module may be employed as long as it can detect both the first light and the second light. When the first light has a wavelength of 1550 nm and the second light has a wavelength of 1310 nm, for example, a light receiving module having a light receiving element using GaAs may be employed as the light receiving section.

When the first light or the second light enters the light-receiving element (light-receiving element 51 to be described later) of the light-receiving module 5 through the top surface of the trapezoidal prism 2, the light-receiving module 5 outputs an electrical signal according to the intensity of the incident light. The electrical signal output from the light receiving module 5 is input to the control circuit 6.

The control circuit 6 drives the first light emitting element 3-1, the second light emitting element 3-2, and the light receiving module 5, and calculates the concentration of glucose in the blood of the living body based on the intensities of the first and second lights detected by the light receiving module 5. As a configuration for this purpose, the control circuit 6 includes a processor 11 and a memory 12.

The memory 12 stores in advance correspondence information 13 that records the correspondence (relationship) between the intensity of the first light, the intensity of the second light, and the concentration of glucose in the blood.

For example, the higher the concentration of glucose in the blood, the more the first light is absorbed by glucose. The correspondence information 13 sets forth the relationship between the intensity of the first light and the concentration of glucose in the blood such that the greater the amount of absorption of the first light, that is, the weaker the intensity of the received first light, the higher the concentration of glucose in the blood is calculated.

Furthermore, the thicker the skin (particularly the epidermis) of the living body, the more the amount of first light and second light absorbed by albumin increases. Thus, the correspondence information 13 sets forth the relationship between the intensity of the second light and the concentration of glucose in the blood in order to calibrate for the loss of the first light by the skin (particularly the epidermis) such that the larger the amount of second light absorbed, that is, the weaker the intensity of the second light, the lower the concentration of glucose in the blood is calculated.

Note that the relationship between the intensity of the first light, the intensity of the second light, and the concentration of glucose in the blood is determined, for example, by experiment. In one example, for a large number of subjects, the concentration of glucose in the blood is measured by blood sampling, the intensity of the first light is detected, and the intensity of the second light is detected. Then the correspondence information 13 is generated by investigating and analyzing the relationship between the measured concentration of glucose, the intensity of the detected first light, and the intensity of the detected second light, and is stored in the memory 12.

Note that the correspondence information 13 may be obtained by calculation.

The processor 11 causes the first light emitting element 3-1 and the second light emitting element 3-2 to emit respective light at different timings, and acquires a signal indicating the intensity of the first light and a signal indicating the intensity of the second light, respectively output from the light receiving module 5. Then, the processor 11 calculates the concentration of glucose in the blood based on these acquired signals and the correspondence information 13.

Note that the processor 11 may be configured, for example, by a CPU (Central Processing Unit), or may be realized by other hardware (circuit) using an FPGA (Field-Programmable Gate Array) or an ASIC (Application Specific Integrated Circuit).

Furthermore, the control circuit 6 can be appropriately equipped with an amplifier circuit that amplifies the signals from the light receiving module 5, and an analog-to-digital conversion circuit that converts the signals into digital values.

The control circuit 6 sends the concentration of glucose in the blood obtained by the calculation to the display device 7 as a measurement result.

The display device 7 is, for example, an LCD (Liquid Crystal Display) or an OELD (Organic Electroluminescent Display). The display device 7 displays the concentration of glucose in the blood sent from the control circuit 6 in a manner that is visible to the user. The display device 7 may display the concentration as numerical information, or may display it as a graph, a bar, or the like.

Note that the glucose measuring device 1 does not necessarily include the display device 7. The glucose measuring device 1 may include a speaker instead of the display device 7, and may output the concentration of glucose in the blood as audio information through the speaker.

Furthermore, the glucose measuring device 1 may include a wired or wireless interface that can be connected to an external device such as a personal computer, and the control circuit 6 may transmit the concentration of glucose in the blood obtained by calculation to the external device via the interface.

### (Configuration of Trapezoidal Prism 2 and Arrangement of Components Around Trapezoidal Prism 2 According to an Embodiment)

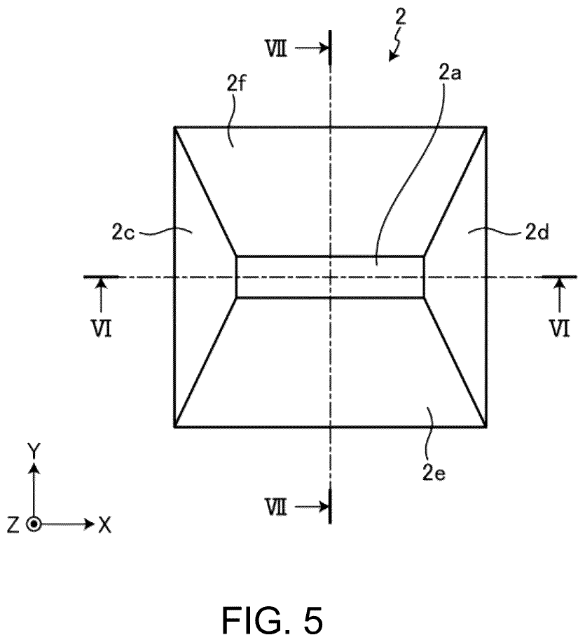

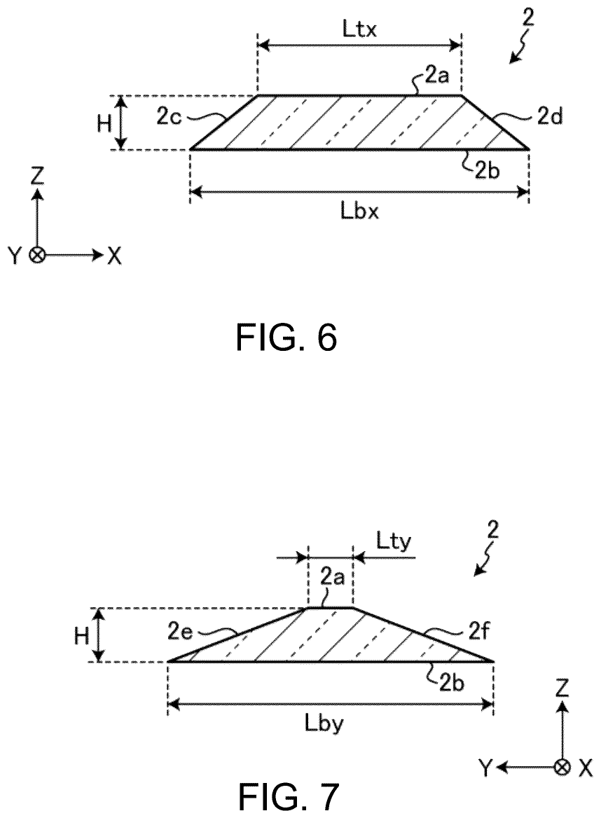

FIG. 5 is an external view of the trapezoidal prism 2 according to an embodiment viewed from the top side. FIG. 6 is a cross-sectional view of the trapezoidal prism 2 according to the embodiment taken along the cutting line VI-VI shown in FIG. 5. FIG. 7 is a cross-sectional view of the trapezoidal prism 2 according to the embodiment taken along the cutting line VII-VII shown in FIG. 5. The shape of the trapezoidal prism 2 will be explained with reference to these figures. Note that in these figures and some subsequent figures, the direction from the bottom surface to the top surface of the trapezoidal prism 2 is defined as the Z direction. The direction perpendicular to the Z direction and parallel to a certain side of the bottom (and top) of the trapezoidal prism 2 is defined as the X direction. The direction perpendicular to the Z direction and the X direction and parallel to another side of the bottom surface (and top surface) of the trapezoidal prism 2 is defined as the Y direction.

The surface 2a is the top surface on which the light receiving module 5 is placed, and the surface 2b is the bottom surface that is pressed against the human body 150. Hereinafter, the surface 2a will be referred to as the top surface 2a, and the surface 2b will be referred to as the bottom surface 2b. Surfaces 2c, 2d, 2e, and 2f are side walls. The surfaces 2c, 2d, 2e, and 2f are referred to as side walls 2c, 2d, 2e, and 2f. The first light is incident on the side wall 2c. The second light is incident on the side wall 2d.

Note that the top surface 2a is an example of the first surface of the light collecting section, and is an example of the first surface of the first optical element. The bottom surface 2b is an example of the second surface of the light collecting section, and is an example of the second surface of the first optical element. The side walls 2c and 2d are examples of wall surfaces of the light collecting section. The side wall 2c is an example of the third surface of the first optical element.

According to a simulation conducted by the inventor, when the area of the bottom surface exceeds a range of 8 mm in diameter centered on the light irradiation position, the reflected light within the dermis becomes zero, and so it is not efficient or effective to collect reflected light outside of the diameter of 8 mm. For this reason, by setting the dimensions of the bottom surface 2b, that is, the length Lbx of the bottom surface 2b in the X direction and the length Lby of the bottom surface 2b in the Y direction, to be 8 mm, for example, the bottom surface 2b can receive all the light that is reflected back within the dermis.

The length Lbx of the bottom surface 2b in the X direction is longer than the length Ltx of the top surface 2a in the X direction, and the length Lby of the bottom surface 2b in the Y direction is longer than the length Lty of the top surface 2 in the Y direction. That is, the trapezoidal prism 2 has a shape whose cross section is enlarged from the top surface 2a where the light receiving module 5 is placed toward the bottom surface 2b that is pressed against the human body 150. Therefore, the trapezoidal prism 2 can collimate the light that has entered from the bottom surface 2b and returned from the human body 150 onto the top surface 2a by multiple reflections or the like.

As mentioned above, the light receiving module 5 is arranged on the top surface 2a.

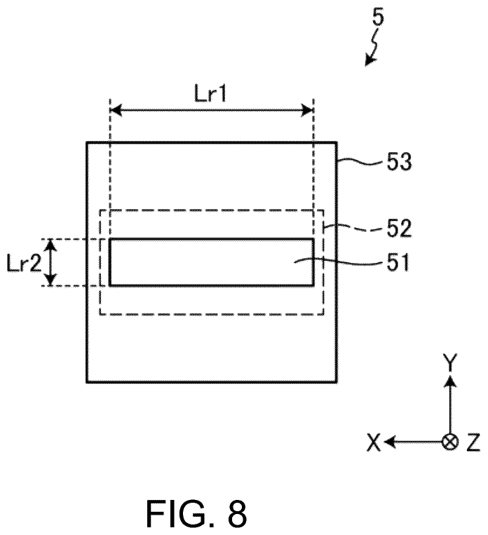

FIG. 8 is a schematic diagram showing an example of the configuration of the light receiving module 5 according to the embodiment.

According to the example shown in FIG. 8, the light receiving module 5 has a structure in which a light receiving element 51 and a circuit 52 are sealed with a resin 53. For example, the light-receiving element 51 is a photodiode, and the circuit 52 is an IC in which a driving/arithmetic circuit for the light-receiving element 51 is configured in one chip, and a transparent plate such as a glass plate is placed on the light-receiving surface side of the light-receiving element 51. The surface of the glass plate is exposed and integrally molded with resin 53.

The light receiving element 51 is exposed on a surface of the light receiving module 5, and the circuit 52 is arranged on the opposite side to the side where the light receiving element 51 is exposed. The light receiving module 5 is arranged such that the exposed portion of the light receiving element 51 or the glass plate faces the top surface 2a. That is, FIG. 8 is a view of the contact surface of the light receiving module. That is, this is a diagram of the light receiving module 5 in the arranged state as viewed from the negative side in the Z direction.

The light receiving element 51 converts the first light and the second light into electrical energy by photoelectric conversion. The light receiving element 51 is made of GaAs, for example. The circuit 52 acquires electrical energy from the light receiving element 51 and outputs the acquired electrical energy as an electrical signal. Thereby, the light receiving module 5 can output an electric signal according to the intensity of the received light. Note that an optical IC made of Si semiconductor may be used instead.

In order to completely detect the light that is collected on the top surface 2a of the trapezoidal prism 2 and emitted from the top surface 2a, the light receiving module 5 is configured such that the light receiving surface, that is, the light receiving element 51 is placed to cover the top surface 2a of the trapezoidal prism 2. For example, the light receiving module 5 having the light receiving element 51 that has a length Lr1 of one side that is longer than or equal to the length Ltx of the top surface 2a in the X direction, and a length Lr2 of another side that is longer than or equal to the length Lty of the top surface 2a in the Y direction is selected as the light receiving section of the glucose measuring device 1. Such a light receiving module 5 is arranged on the top surface 2a side of the trapezoidal prism 2 so that the side having a length of Lr1 is parallel to the X direction, and the side having a length of Lr2 is parallel to the Y direction so as to cover the top surface 2a of the trapezoidal prism 2 with the light receiving element 51.

In general, the smaller the light receiving surface area of a light receiving module is, the better the response speed to incident light is, and the cheaper it is. In the embodiment, the top surface 2a is smaller than the bottom surface 2b in terms of area. Therefore, the designer can select a light-receiving module that has a light-receiving element with a small area and is excellent in terms of cost and reaction speed as the light-receiving section of the glucose measuring device 1.

Note that the designer may select a light-receiving module whose light-receiving surface area is as small as possible as the light-receiving section of the glucose measuring device 1, and the top surface 2a may be designed according to the dimensions of the bottom surface 2b and the dimensions of the light-receiving element of the selected light-receiving module.

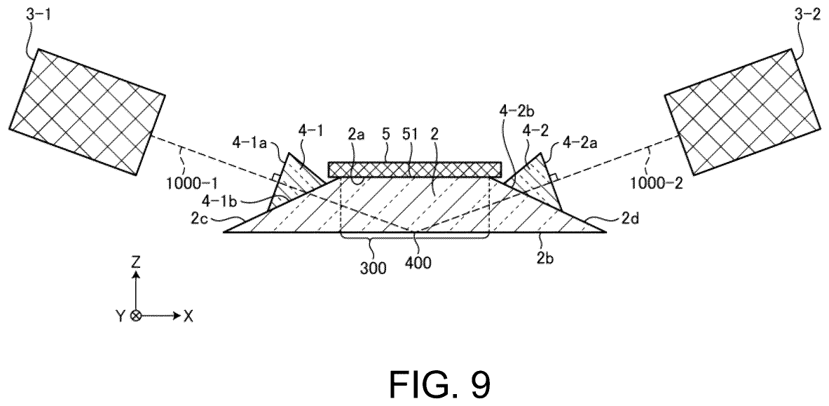

FIG. 9 is a schematic diagram for explaining an example of the arrangement relationship between the trapezoidal prism 2 according to the embodiment and components around it. In FIG. 9, 1000-1 indicates the optical path of the first light emitted from the first light emitting element 3-1, and 1000-2 indicates the optical path of the second light emitted from the second light emitting element 3-2.

As described above, the intensity of the light reflected within the dermis and returned is strongest at the light irradiation position and decreases as it moves away from the light irradiation position. Therefore, the attitudes of the first light emitting element 3-1 and the second light emitting element 3-2 are determined such that the first light and the second light are emitted from the center position 400 of the area 300 in front of the top surface 2a on the bottom surface 2b to the human body 150. When the bottom surface 2b is pressed against the human body 150, the light emitted to the human body 150 from the position 400 returns strongly from the position 400. Since the light receiving module 5 is located directly in front of the position 400 where the light returns strongly, it can receive a large amount of the return light from the human body 150.

When light enters an object, loss occurs due to reflection of the light on the surface of the object. The amount of light lost upon incidence increases as the angle between the light and the surface of the object deviates from the perpendicular.

A first triangular prism 4-1 is arranged on the side wall 2c on which the first light enters in order to suppress this loss due to reflection of the first light. Specifically, the shape and position of the first triangular prism 4-1 are determined such that the first light is perpendicularly incident on a wall surface 4-la of the three side walls of the first triangular prism 4-1, and that the other wall surface 4-1b is in close contact with the side wall 2c. The refractive index of the first triangular prism 4-1 is equal to that of the trapezoidal prism 2. The wall surface 4-1b and the side wall 2c are connected to each other by, for example, an adhesive. In such a case, the refractive index of the adhesive is equal to the refractive index of the trapezoidal prism 2 and the first triangular prism 4-1.

Therefore, the first light enters the wall surface 4-1a of the first triangular prism 4-1 perpendicularly, and then travels straight through the boundary between the wall surface 4-1b and the side wall 2c, and is emitted to and enters the human body 150 from the position 400.

Note that the first triangular prism 4-1 is an example of the second optical element. The wall surface 4-la is an example of the fourth surface. The wall surface 4-1b is an example of the fifth surface.

A second triangular prism 4-2 is arranged on the side wall 2d on which the second light enters in order to suppress the above-mentioned loss due to reflection of the second light. Specifically, the shape and position of the second triangular prism 4-2 are determined such that the second light is perpendicularly incident on a wall surface 4-2a of the three side walls of the second triangular prism 4-2, and the other wall surface 4-2b is in close contact with the side wall 2d. The refractive index of the second triangular prism 4-2 is equal to that of the trapezoidal prism 2. The wall surface 4-2b and the side wall 2d are connected to each other by, for example, an adhesive. In such a case, the refractive index of the adhesive is equal to the refractive index of the trapezoidal prism 2 and the second triangular prism 4-2.

Therefore, the second light enters the wall surface 4-2a of the second triangular prism 4-2 perpendicularly, and then travels straight through the boundary between the wall surface 4-2b and the side wall 2d, and is emitted to and enters the human body 150 from the position 400.

The height H of the trapezoidal prism 2, that is, the distance between the bottom surface 2b and the top surface 2a, is determined based on the workability of the trapezoidal prism 2 and the light utilization efficiency. The light utilization efficiency is the ratio of the intensity of light incident on the light receiving element 51 to the intensity of light emitted by the light emitting element (first light emitting element 3-1 or second light emitting element 3-2).

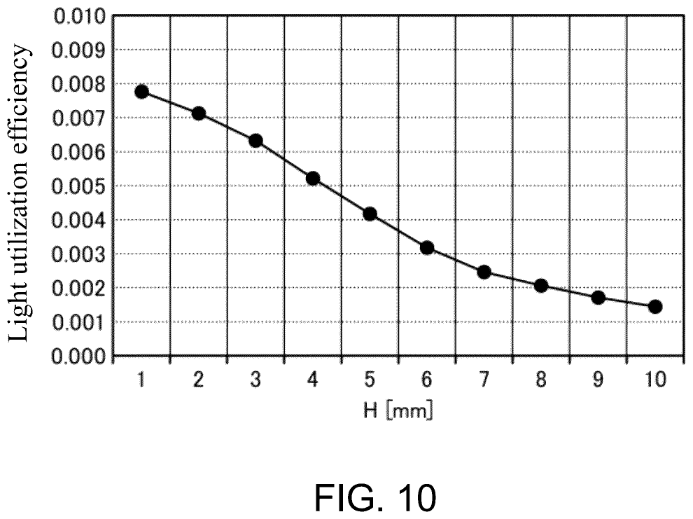

FIG. 10 is a diagram showing an example of the relationship between the height H of the trapezoidal prism 2 and the light utilization efficiency according to the embodiment. From this figure, it can be seen that the lower the height H is, the better the light usage efficiency is. For this reason, the height H is preferably 4 mm or less. However, if the height H is too low, positioning becomes difficult, so it is preferable that the height H is 2 mm or more, for example.

Note that the lower the height H, the more acute the angle formed by the optical axis of each light emitting element 3-1, 3-2 with respect to the bottom surface 2b, and as a result, the angle of incidence of each light on the side walls 2c, 2d deviates from perpendicular. However, in the embodiment, the first triangular prism 4-1 and the second triangular prism 4-2 are provided so that the angle of incidence of light to the medium of the optical element (that is, the medium of the trapezoidal prism 2 and each of the triangular prisms 4-1 and 4-2) is made perpendicular. Therefore, even if the angle of incidence of each light beam on the side walls 2c and 2d deviates from perpendicular as a result of reducing the height H, loss due to reflection when each light beam enters the medium of the optical element is suppressed. Therefore, the height H of the trapezoidal prism 2 can be reduced. As described above, in terms of light utilization efficiency, the lower the height H is, the better. Therefore, by employing the triangular prisms, the light utilization efficiency can be increased.

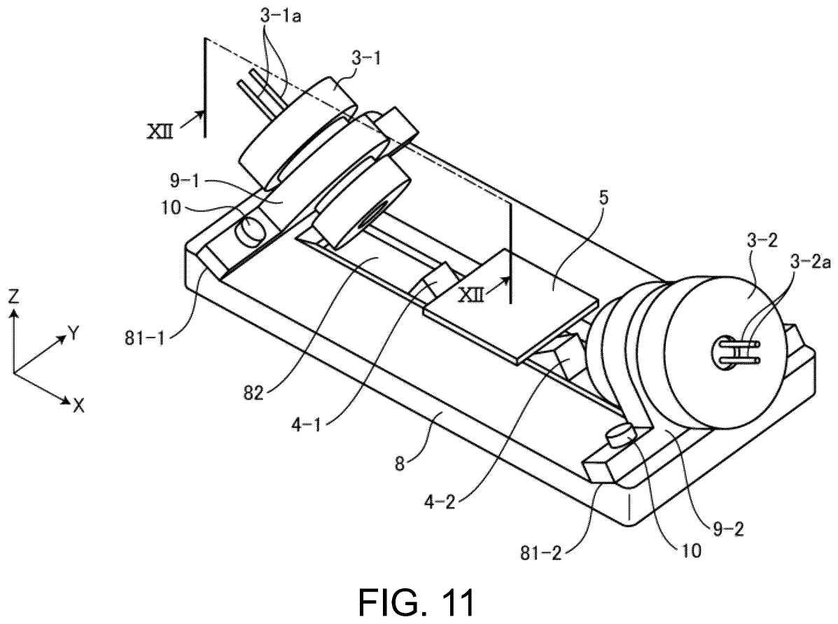

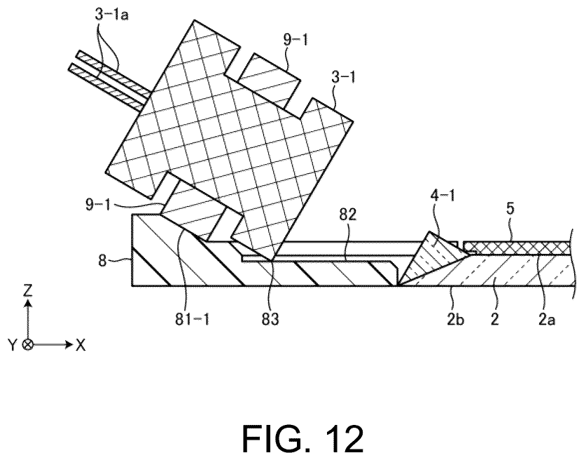

FIG. 11 is a perspective view showing an example of a structure that supports the trapezoidal prism 2 and surrounding components according to an embodiment. FIG. 12 is a cross-sectional view of the structure shown in FIG. 11 taken along cutting line XII-XII.

As shown in FIGS. 11 and 12, the trapezoidal prism 2, the first triangular prism 4-1, the second triangular prism 4-2, the first light emitting element 3-1, the second light emitting element 3-2, and the light receiving element 51 are fixed to a pedestal 8 included in the glucose measuring device 1. The pedestal 8 is provided with a groove 82 extending in the X direction, and the trapezoidal prism 2 is fitted into the bottom of the groove 82 at the center in the X direction.

The first triangular prism 4-1, the second triangular prism 4-2, and the light receiving module 5 are attached to the trapezoidal prism 2.

A first slope 81-1 is provided on the pedestal 8 on the negative side of the groove 82 in the X direction. The body of the first light emitting element 3-1 is non-slidably supported by a first metal fitting 9-1, and the first metal fitting 9-1 is fixed to the first slope 81-1 with a screw 10. Therefore, the posture of the first light emitting element 3-1 is fixed so that the first light emitted from the first light emitting element 3-1 enters the trapezoidal prism 2 via the first triangular prism 4-1.

A part of the tip of the first light emitting element 3-1 contacts a position 83 at the bottom of the groove 82. Therefore, the first light emitting element 3-1 is firmly positioned by the first metal fitting 9-1 and the bottom of the groove 82.

Similarly, a second slope 81-2 is provided on the pedestal 8 on the positive side of the groove 82 in the X direction. The body of the second light emitting element 3-2 is non-slidably supported by a second metal fitting 9-2, and the second metal fitting 9-2 is fixed to the second slope 81-2 with a screw 10. Therefore, the posture of the second light emitting element 3-2 is fixed so that the second light emitted from the second light emitting element 3-2 enters the trapezoidal prism 2 via the second triangular prism 4-2.

Note that a portion of the tip of the second light emitting element 3-2 also comes into contact with the bottom of the groove 82. Therefore, the second light emitting element 3-2 is firmly positioned by the second metal fitting 9-2 and the bottom of the groove 82.

### (Operation of Glucose Measuring Device 1 According to an Embodiment)

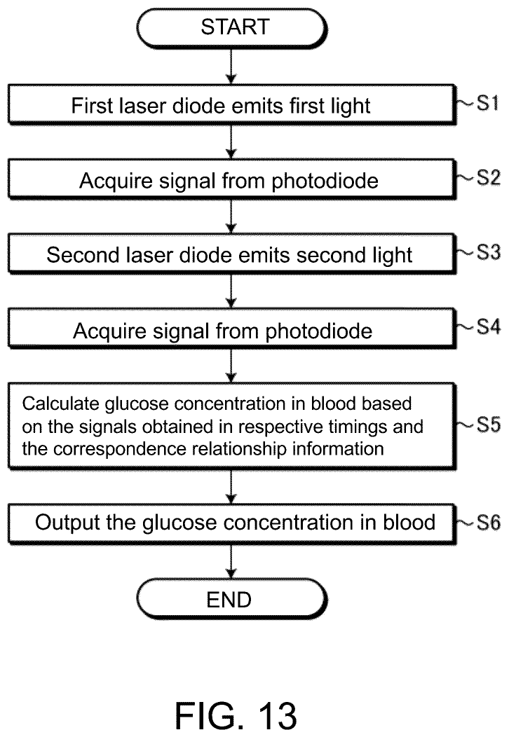

FIG. 13 is a flowchart illustrating an example of the operation of the glucose measuring device 1 according to an embodiment. First, the control circuit 6 (more specifically, the processor 11) causes the first light emitting element 3-1 to emit light (S1). During the period in which the first light emitting element 3-1 emits light, the control circuit 6 acquires the electrical signal output from the light receiving module 5 (S2). Thereby, the control circuit 6 can acquire the intensity of the first light irradiated onto the surface of the living body and returned.

Subsequently, the control circuit 6 stops the first light emitting element 3-1 from emitting light, and causes the second light emitting element 3-2 to emit light (S3). During the period in which the second light emitting element 3-2 emits light, the control circuit 6 acquires the electrical signal output from the light receiving module 5 (S4). Thereby, the control circuit 6 can acquire the intensity of the second light irradiated onto the surface of the living body and returned.

Subsequently, the control circuit 6 calculates the concentration of glucose in the blood based on the signal indicating the intensity of the first light obtained through the processing in S2, the signal indicating the intensity of the second light obtained through the processing in S4, and the correspondence relationship information 13 stored in the memory 12 (S5).

Then, the control circuit 6 outputs the concentration of glucose in the blood obtained by the calculation to the display device 7 (S6), and the operation of the glucose measuring device 1 ends.

### (Variations of the Light Collecting Section)

The light collecting section applicable to the glucose measuring device 1 is not limited to the trapezoidal prism 2. Variations of the light collecting section will be described below.

For example, when an optical element made of a medium that transmits light used for measurement is used as the light collecting section, the shape of the bottom surface of the light collecting section is not limited to a rectangle. The bottom surface of the light collecting section may have a circular shape centered at the position 400. The side walls 2c and 2d into which light is incident do not have to be flat. The side walls 2c and 2d on which light is incident may be curved surfaces. When the side wall 2c and the side wall 2d are formed of curved surfaces, a flat surface may be provided in a portion where light used for measurement is incident.

When the shape of the bottom surface of the light collecting section is rectangular, the dimensions of the bottom surface of the light collecting section do not necessarily have to be an 8 mm square. The length of each side of the bottom surface of the light collecting section does not have to be equal to 8 mm. If the area of the bottom surface is at least larger than the area of the top surface, it is possible to condense the return light incident on the bottom surface onto the top surface.

Furthermore, the light collecting section is not limited to an optical element made of a medium that transmits light. For example, the light collecting section may be a hood having a through hole.

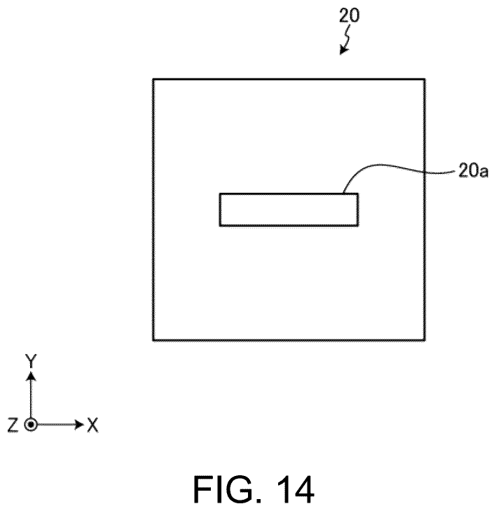

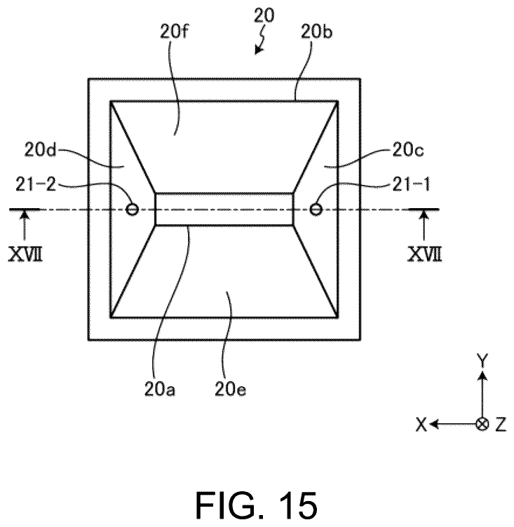

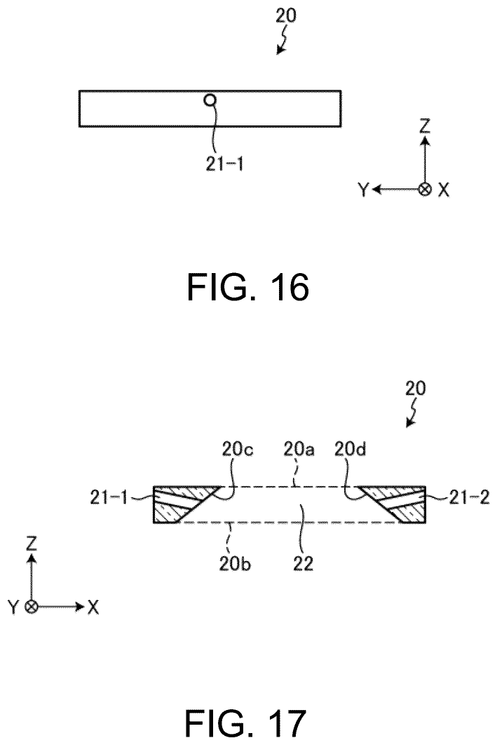

FIG. 14 is a top view of a hood 20 as another example of the light collecting section according to an embodiment. FIG. 15 is a bottom view of the hood 20 as another example of the light collecting section according to the embodiment. FIG. 16 is a side view of the hood 20 as another example of the light collecting section according to the embodiment. FIG. 17 is a cross-sectional view of the hood 20 as another example of the light collecting section according to the embodiment, taken along the cutting line XVII-XVII shown in FIG. 15.

The hood 20 includes a first opening 20a, a second opening 20b, and a through hole 22 that connects the first opening 20a and the second opening 20b. Both the first opening 20a and the second opening 20b are rectangular.

The opening area of the first opening 20a is smaller than the opening area of the second opening 20b. The first opening 20a is covered by the light receiving element 51, like the top surface 2a of the trapezoidal prism 2. The dimensions of the second opening 20b are determined based on the same criteria as the bottom surface 2b of the trapezoidal prism 2 so that it can receive as much light as possible that is irradiated onto the human body 150 and returns.

The through hole 22 has inner walls (wall surfaces) 20c, 20d, 20e, and 20f. The inner wall 20c is provided with a through hole 21-1 through which the first light emitted from the first light emitting element 3-1 passes. The inner wall 20d is provided with a through hole 21-2 through which the second light emitted from the second light emitting element 3-2 passes.

The hood 20 is constructed from a solid material such as metal, glass, or resin. The inner walls 20c, 20d, 20e, and 20f of the hood 20 have a structure that reflects light used in measurement. For example, when the hood 20 is made of metal, the inner walls 20c, 20d, 20e, and 20f of the hood 20 are processed to have a mirror surface. When the hood 20 is made of a material other than metal, the inner walls 20c, 20d, 20e, and 20f of the hood 20 are made of a reflective material such as aluminum or gold and have a mirror-like structure. Note that the examples of structures that reflect light provided on the inner walls 20c, 20d, 20e, and 20f of the hood 20 are not limited to these.

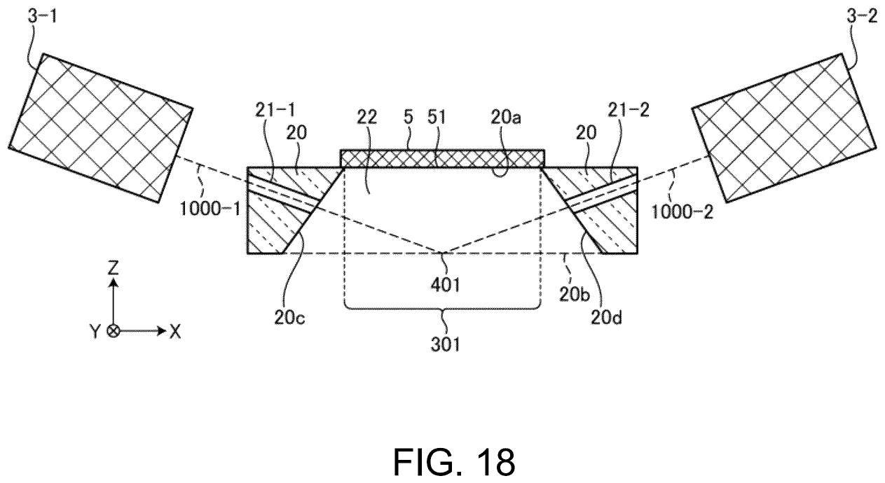

FIG. 18 is a schematic diagram for explaining an example of the arrangement relationship between the hood 20 as another example of the light collecting section according to the embodiment and components around the hood 20. In FIG. 18, 1000-1 indicates the optical path of the first light emitted from the first light emitting element 3-1, and 1000-2 indicates the optical path of the second light emitted from the second light emitting element 3-2.

As shown in FIG. 18, the attitudes of the first light emitting element 3-1 and the second light emitting element 3-2 and the positions and directions of the through holes 21-1 and 21-2 are determined such that the first light and the second light are emitted to the human body 150 from a central position 401 of a region 301 in front of the first opening 20a in the second opening 20b.

When the human body 150 is pressed against the second opening 20b, the intensity of the light that is irradiated onto the human body 150 at the position 401, reflected within the dermis, and is returned is the strongest at the position 401. Since the intensity of the returned light is maximum at the center position 401 of the area 301 in front of the first opening 20a in the second opening 20b, the light receiving module 5 provided directly in front of the position 401 receives the returned light in a large amount.

In this way, it is possible to use a hood in which a through hole having a structure that reflects light is provided in the inner walls as the light collecting section.

Note that each opening of the hood as a light collecting section is not limited to a rectangular shape. At least one of the two openings of the hood serving as the light collecting section may be circular.

Regarding the above-mentioned hood 20, the first opening 20a is an example of the first surface of the light collecting section. The second opening 20b is an example of the second surface of the light collecting section. The inner walls 20c, 20d, 20e, and 20f are examples of wall surfaces of the light collecting section. The through hole 22 is an example of a first through hole of the light collecting section. The through hole 21-1 is an example of a second through hole of the light collecting section.

As seen above, the light collecting section according to the embodiments can be modified in various ways.

### (Variations of the Light Used)

In the above descriptions, the glucose measuring device 1 irradiates the human body 150 with the first light and the second light, and based on the intensity of the returned light of the first light and the intensity of the returned light of the second light, the concentration of glucose in the blood was measured.

Alternatively, the glucose measuring device 1 may be configured to measure the concentration of glucose in blood based only on the first light. That is, it is possible to omit the second light emitting element 3-2 and the second triangular prism 4-2 from the glucose measuring device 1. When the hood 20 is used as a light collecting section, the through hole 21-2 can be omitted.

Furthermore, the glucose measuring device 1 may be configured to be able to emit three or more lights including the first light and the second light. For example, the glucose measuring device 1 may be configured so that light emitted by another light emitting element is incident from the side wall 2e or the side wall 2f. When the hood 20 is used as the light collecting section, a through hole may be provided in the inner wall 20e or 20f to allow light emitted by another light emitting element to pass therethrough.

Furthermore, in the glucose measuring device 1, the wavelength of the first light is not limited to a wavelength selected from the range of 1500 nm to 1700 nm, as long as it has the property of being easily absorbed by glucose. Further, the wavelength of the second light is not limited to a wavelength selected from the range of 1000 nm to 1400 nm, as long as it is absorbed to some extent by the main components of the human body 150 other than glucose and has flat absorption characteristics for both the components and glucose.

Furthermore, when a plurality of lights are used in the glucose measuring device 1, the glucose measuring device 1 may be configured to irradiate the human body 150 with two or more lights having the same wavelength from different wall surfaces or inner walls.

As described above, the glucose measuring device 1 as the measuring device according to the embodiments includes the first light emitting element 3-1 as the first light emitting section that emits the first light, a trapezoidal prism 2 as a light collecting section that has the top surface 2a as a first surface, the bottom surface 2b as a second surface that faces the first surface and that has a larger area than the first surface, and the side wall 2c as a first wall surface that connects the first surface and the second surface and that passes the first light toward the second surface; the light receiving module 5 as a light receiving section disposed on the first surface and receiving first light irradiated onto a living body from the second surface and returned from the living body; and the control circuit 6 as a calculation section that receives the output from the light receiving section and calculates numerical information regarding the living body.

For example, in the technique described in Japanese Patent Application Laid-open No. JP 2017-196211 A, the irradiation position and the position receiving the return light from the human body are separated from each other. Therefore, the light that strongly returns to the irradiation position cannot be received, resulting in low light utilization efficiency.

On the other hand, according to the embodiments of the present invention, the bottom surface 2b as the second surface can receive the light that strongly returns at the irradiation position so that a high light utilization efficiency can be obtained.

Further, according to the embodiments, the first light is configured to enter the trapezoidal prism 2 as the light collecting section from the side wall 2c as the wall surface instead of the top surface 2a as the first surface. Therefore, the light receiving module 5 can be arranged so as to cover the top surface 2a. By arranging the light receiving module 5 so as to cover the top surface 2a, it is possible to detect all of the light that is collected on the top surface 2a and emitted from the top surface 2a. Therefore, it is possible to further improve the light usage efficiency.

Further, according to the embodiments, the first light is emitted from the center position 400 of the area 300 in front of the top surface 2a as the first surface on the bottom surface 2b as the second surface.

Since the light receiving module 5 is placed directly in front of the position 400 where the light returns strongly, it is possible to further improve the light utilization efficiency.

Note that the position from which the light used for measurement is emitted may be shifted from the center of the region 300. Even if the position where the light is emitted is shifted from the center of the area 300, as long as the position where the light is emitted is within the area 300, the light receiving module 5 can receive the light strongly returning from the human body 150.

Furthermore, the position from which the light is emitted may be shifted from the region 300. Even if the position where the light is emitted is shifted from the area 300, the light that strongly returns from the human body 150 is collected on the top surface 2a as the first surface due to multiple reflections, and can be received by the light receiving module 5 from the top surface 2a.

Note that the trapezoidal prism 2 is an example of the light collecting section, and includes a top surface 2a as a first surface, a bottom surface 2b as a second surface, and a side wall 2c as a first wall surface. This is also an example of a first optical element made of a medium that transmits light used for measurement. When this first optical element is applied as a light collecting section, the glucose measuring device 1 as the measuring device according to the embodiments further has a first triangular prism 4-1 as a second optical element made of a medium having the same refractive index as the first optical element. The first triangular prism 4-1 as the second optical element has a wall surface 4-la as a fourth surface that receives the first light perpendicularly, and a wall surface 4-2b as a fifth surface that is in contact with the side wall 2c as the third surface and that allows the first light received on the wall surface 4-la as the fourth surface to pass through the side wall 2c as the third surface.

Therefore, it is possible to suppress the amount of light loss due to reflection when the light used for measurement is incident on the medium of the first and second optical elements. Since light loss due to reflection is suppressed, it is possible to further improve light utilization efficiency.

Note that the light collecting section may be the food 20 that has the first opening 20a as the first surface, the second opening 20b as the second surface, and inner walls 20c, 20d, 20e, and 20f as the first wall surfaces. The inner walls 20c, 20d, 20e, and 20f of the hood 20 as the light collecting section are structured to reflect light used for measurement, and the inner wall 20c has the through hole 21-1 as the second through hole through which the first light passes.

Further, the glucose measuring device 1 as the measuring device according to the embodiment includes the second light emitting element 3-2 as the second light emitting section that emits second light. The wall surface of the light collecting section allows the second light to pass toward the second surface by transmission in the case of the trapezoidal prism 2, and by the through hole 21-2 in the case of the hood 20. The light receiving module 5 as the light receiving section is configured to receive second light that is irradiated onto the human body 150 from the second surface and returned from the human body 150.

Therefore, for example, when light with a wavelength selected from the range of 1500 nm to 1700 nm is used as the first light, and light with a wavelength selected from the range of 1000 nm to 1400 nm is used as the second light, it is possible to measure glucose concentration with high accuracy by reducing the influence of light absorption by albumin, which is the main component of the skin. Note that, as described above, the first light and the second light are not limited to these.

Furthermore, the optical measuring device according to the present invention can be applied to a pulse wave measuring device that measures pulse waves. In such a case, for example, green light, which is easily absorbed by hemoglobin, may be used as the first light. Note that the first light used in the pulse wave measuring device is not limited to this. The pulse wave measuring device may be configured to irradiate the human body 150 with two or more lights having different wavelengths or the same wavelength.

The optical measuring device according to the present invention can be implemented in a wristwatch, or can also be configured as a wristwatch-type device. The optical measuring device according to the present invention can also be applied to a measuring device that measures the concentration of other specific components contained in blood.

According to an aspect of the present invention, it is possible to provide a measuring device with improved light utilization efficiency.

Although various embodiments of the present invention have been described, these embodiments are presented as examples and are not intended to limit the scope of the invention. This novel embodiment can be implemented in various other forms, and various omissions, substitutions, and changes can be made without departing from the gist of the invention. These embodiments and its modifications are included within the scope and gist of the invention, as well as within the scope of the invention described in the claims and its equivalents. It will be apparent to those skilled in the art that various modifications and variations can be made in the present invention without departing from the spirit or scope of the invention. Thus, it is intended that the present invention cover modifications and variations that come within the scope of the appended claims and their equivalents. In particular, it is explicitly contemplated that any part or whole of any two or more of the embodiments and their modifications described above can be combined and regarded within the scope of the present invention.

What is claimed is:

1. A measuring device, comprising:
a first light emitting section that emits first light;
a light collecting section that has a first surface, a second surface that faces the first surface and that has a larger area than the first surface, and a wall surface that connects the first surface and the second surface and that allows the first light to pass therethrough;
a light receiving section that is disposed on the first surface to receive the first light irradiated onto a living body from the second surface and returned from the living body; and
a calculation section that receives an output from the light receiving section to calculate numerical information regarding the living body.

2. The measuring device according to claim 1, wherein the first light is emitted from an area in the second surface that directly faces the first surface.

3. The measuring device according to claim 2, wherein the first light is emitted from a center of said area in the second surface.

4. The measuring device according to claim 1, wherein the light collecting section is a first optical element made of a first medium that transmits the first light and that has the first surface, the second surface, and a third surface that is the wall surface.

5. The measuring device according to claim 4, further comprising a second optical element that is made of a second medium having the same refractive index as the first medium, and that has a fourth surface that perpendicularly receives the first light and a fifth surface that contacts the wall surface and that allows the first light received by the fourth surface to pass through the wall surface.

6. The measuring device according to claim 1,
wherein the light collecting section has a first opening as the first surface, a second opening as the second surface, a first through hole that penetrates from the first opening to the second opening, and an inner wall of the first through hole as the wall surface, and
wherein the inner wall is structured to reflect the first light, and has a second through hole therein to allow the first light to pass through.

7. The measuring device according to claim 1, further comprising a second light emitting section that emits second light,
wherein the light collecting section allows the second light to pass through towards the second surface, and
wherein the light receiving section receives the second light irradiated onto the living body from the second surface and returned from the living body.

8. The measuring device according to claim 7, wherein a wavelength of the second light is different from a wavelength of the first light.

9. The measuring device according to claim 1, wherein the numerical information is a concentration of glucose in blood in the living body.

10. The measuring device according to claim 1, wherein the numerical information is information on a pulse wave of the living body.

11. A measuring device, comprising:
a first prism that has a bottom surface, a top surface having a smaller area than the bottom surface, and a first inclined wall surface extending between the top surface and the bottom surface and forming an acute angle with the bottom surface;
a first triangular prism that has a first side surface that is in surface contact with the first inclined wall surface of the first prism, and a second side surface forming an angle with the first side surface, an angle of the second side surface with respect to the bottom surface being greater than said acute angle;
a first light emitting element arranged such that light from the first light emitting element enters the second side surface of the first triangular prism perpendicularly or near perpendicularly, and that the light that has passed through the first triangular prism is incident on the first prism; and
a light-receiving module having a light-receiving element whose light-receiving surface covers the top surface of the first prism.

12. The measuring device according to claim 11, further comprising:
a second triangular prism attached to a second inclined wall surface of the first prism, the first prism having a rectangular top surface and a rectangular bottom surface and having said second inclined wall surface opposite to the first inclined wall surface thereof; and
a second light emitting element having an optical path that is incident on a second side surface of the second triangular prism at an angle greater than said acute angle.

13. A wristwatch or a wristwatch-type measuring device in which the measuring device according to claim 12 is embedded, wherein said optical path of the second light emitting element and the second side surface of the second triangular prism are set to be perpendicular to each other.

14. The measuring device according to claim 11, further comprising:
a pedestal with a rectangular groove; and
a prism module in which the first prism and the first triangular prism are integrated, and the second side surface of the first triangular prism is disposed on a short side of the rectangular groove and fitted into the groove,
wherein a body of the first light emitting element is provided at an angle with respect to the pedestal at an outside of the short side of the groove, and a tip end of the first light emitting element is made to contact the pedestal so as to fix the first light emitting element to the pedestal.
